(19) United States
(12) Patent Application Publication
(10) Pub. No.: US 2006/0013827 A1
(43) Pub. Date: Jan. 19, 2006

(54) Method of diagnosing FIV

(76) Inventors: Dorothee Bienzle, Ontario (CA); Felipe Reggeti, Ontario (CA)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/155,232

(22) Filed: Jun. 17, 2005

Related U.S. Application Data
(60) Provisional application No. 60/580,731, filed on Jun. 21, 2004.

(51) Int. Cl. A61K 39/12; C12Q 1/70; C12Q 1/68

(52) U.S. Cl. 424/199.1; 435/5; 435/6

(57) ABSTRACT

The present invention is directed to a method for diagnosing feline immunodeficiency virus (FIV) infection. The method involves the PCR amplification of a DNA sample obtained from a cat to be diagnosed. The PCR amplification utilizes a first gag-specific DNA primer derived from the gag gene region of an FIV genome and a second FIV-specific DNA primer that binds to a region of an FIV genome suitably spaced from the first primer to permit DNA amplification to occur. The use of such primers advantageously allows for the exponential amplification of proviral DNA of any FIV subtype, thereby providing a universal means to diagnose FIV infection.

### FIELD OF THE INVENTION

The present invention relates to a method of diagnosing feline immunodeficiency virus (FIV) infection using a novel polymerase chain reaction (PCR) assay. In particular, the present invention relates to a universal FIV diagnostic method utilizing FIV gag-specific primers which permit diagnosis of any FIV infection.

### BACKGROUND OF THE INVENTION

The feline immunodeficiency virus, first identified as a T-lymphotropic virus (Pedersen et al., 1987), is a lentivirus of the family Retroviridae and is related to the human immunodeficiency virus (HIV) (Olmsted et al. 1989a; Olmsted et al., 1989b; Pedersen et al., 1987; Sparkes et al., 1993). Infected cats typically develop CD4+ T lymphocytopenia that leads to immunodeficiency, opportunistic infections, increased occurrence of neoplasia, or a neurological syndrome. Based on genetic, morphological and clinical similarities, FIV has been a valuable model for understanding aspects of HIV pathogenesis and for developing intervening strategies to control infection and disease (Bendinelli et al., 1995; Johnson et al., 1994; Okada et al., 1994; Pedersen et al., 1987).

Lentiviruses, being retroviruses, invade their host organism by attaching to receptors on the extracellular surface of a host cell, after which the viral RNA genome is introduced into the host cell. Through the enzymatic activity of the viral reverse transcriptase, a DNA copy of the viral RNA is made within the host cell, this DNA copy being able to integrate into the host cell's genome. Reproduction of the virus occurs not only through the activity of the host cell's self-replicative machinery, but also through the co-opting of the host cell's transcription and translation mechanisms in order to produce more of the functional virus within the host. Exponential reproduction of the virus within the host cell thus occurs. Lentiviral structure, in terms of the relative ordering and known function of the various major genes in lentiviral RNA genome has been described in U.S. Pat. No. 5,478,724. Two of the major genes in the lentiviral genome are gag and env, which respectively encode for the structural capsid proteins and viral extra-cellular envelope proteins. Situated at either end of the viral genome and adjacent to the gag and env genes are the long terminal repeats (LTR's), while situated between gag and env are the genes encoding for the nucleic acid binding proteins, the viral protease, reverse transcriptase, the tethering protein, RNase H, and the viral endonuclease. Overall, the lentiviral genome size is approximately 10,000 base pairs, and with respect to FIV, there has been found to be about 30% sequence variation between various viral strains for the env gene and about 10% sequence variation for the remainder of the FIV genes. Kakinuma et al. (1995) provide a detailed analysis of the nucleotide and amino acid differences between the North American, European and Japanese strains of FIV.

FIV is distributed worldwide (Bachmann et al., 1997; Ishida et al., 1989; Nishimura et al., 1998). The prevalence of infection is highly variable ranging from 1% in cats at low risk in the United States and Canada (Yamamoto et al., 1989) to 44% in symptomatic cats in Japan (Hohdatsu et al., 1998), depending upon factors such as age, gender and indoor or outdoor housing. Currently, FIV isolates are classified into 5 different subtypes designated as A, B, C, D and E, based on envelope sequence analysis (Nishimura et al., 1998). Subtype A was found in California and Europe while subtype B was prevalent in the Central and Eastern United States (Sodora et al., 1994). Subtype C has been reported from British Columbia and Ontario, Canada (Bachmann et al., 1997; Sodora et al., 1994); while subtypes D and E have been reported from Japan (Hohdatsu et al., 1996; Hohdatsu et al., 1998; Nishimura et al., 1998) and Argentina (Pecoraro et al., 1996), respectively. Classification into subtypes may be accomplished by different methods including subtype-specific PCR (Nishimura et al., 1998), heteroduplex mobility assay (Bachmann et al., 1997), restriction fragment polymorphism (Hohdatsu et al., 1998), and phylogenetic analysis of proviral DNA sequences (Worobey and Holmes, 1999). Although most studies were based on the sequence variation found in the env gene, comparison of sequences of the gag gene can also be useful, since this gene as well has marked variability among different FIV isolates (Hohdatsu et al., 1998; Kakinuma et al., 1995).

Reports in the literature indicate that co-infection with two different HIV-1 strains might occur with exposure to a second virus shortly after the initial infection, or after the initial infection has been established, a condition termed superinfection (Jost et al., 2002). This superinfection phenomenon was also experimentally induced with FIV subtypes A and B, both in vitro and in vivo (Okada et al., 1994). In another study, consecutive exposure of cats to two different FIV strains resulted in superinfection in one cat and recombination in another cat (Kyaw-Tanner et al., 1994), but recombination has rarely been documented in FIV under natural conditions (Bachmann et al., 1997). This superinfection phenomenon and the potential for viral recombination will be of significance as the development of vaccines against lentiviruses such as HIV and FIV will require knowledge of the viral variants being transmitted in the target population (Lole et al., 1999). Since genetic diversity among viruses is extensive, it has been debated whether a vaccine should include a single virus strain, a broad spectrum of viral variants, or only those that are relevant to a particular geographical area (Gao et al., 1998). In addition, intersubtype recombination might complicate the already difficult task of developing vaccines effective against multiple subtypes (Siepel, 1995). Similar to HIV, the high genetic variability displayed by FIV strains, and the possibility of recombination in geographical areas where more than one subtype is present, are major considerations for developing an effective vaccine. Thus, greater understanding of the genetic diversity of FIV would provide a better rationale for vaccine design, and elucidate mechanisms of retroviral recombination.

Recently, a vaccine against FIV was introduced onto the marketplace (Connell, 2003), however, the general use of the vaccine imposes problems with respect to accurately diagnosing FIV infection cats. Standard assays for detecting the presence of FIV in cats involve the use of antibody detection methods, such as the ELISA technique, which allow for the detection of antibodies generated by the infected cat during its immunological response to viral infection. Problematic, however, is the fact that for FIV-vaccinated cats, an antibody detection assay will generate a false-positive result as these cats will a priori have a high antibody titer due to the vaccination.

In view of the foregoing, it is clear that there is the need to provide a diagnostic method that allows for detection of infection by any FIV subtype in a cat. It is also desirable to provide a method that allows for the diagnosis of cats that are truly FIV-infected versus those uninfected cats that have previously been vaccinated and thus have circulating anti-FIV antibodies. Furthermore, it is also desirable to provide an FIV diagnostic method with sufficient sensitivity to allow for the diagnosis of FIV infection during the period between initial infection and the generation of an immunological response in the infected cat.

### SUMMARY OF THE INVENTION

Accordingly, in one aspect, the present invention provides a method for diagnosing FIV infection in a cat comprising the steps of:
(i) obtaining a DNA-containing sample from the cat;
(ii) extracting the DNA from the sample;
(iii) subjecting the extracted DNA to PCR amplification to form an FIV-specific amplicon, wherein the amplification utilizes a first gag-specific primer which binds to a first region on an FIV genome and a second FIV-specific primer which binds to a second region on an FIV genome, and wherein the first and second regions are spaced such that amplification can occur; and
(iv) determining whether an FIV-specific amplicon is generated during step (iii), wherein the presence of an FIV-specific amplicon is indicative of an FIV infection in a cat.

In another aspect of the present invention, a kit is provided comprising a first FIV gag-specific primer which binds to a first region on an FIV genome and a second FIV-specific primer which binds to a second region on an FIV genome, and wherein the first and second regions are spaced such that amplification can occur.

In another aspect of the present invention, an FIV gag-specific primer is provided selected from the group consisting of: a primer comprising the sequence 5′-AGATACCATGCTCTACAC TGC-3′ (SEQ ID No: 4), a primer comprising the sequence 5′-GGTATATCACCAGGTTCTGCT-3′ (SEQ ID No: 6) and a functionally equivalent primer comprising a nucleotide sequence which exhibits at least about 80% sequence homology with one of SEQ ID No: 4 or SEQ ID No: 6.

These and other aspects of the present invention are described by reference to the following figures in which:

### BRIEF DESCRIPTION OF THE FIGURES

FIG. 1 illustrates the nucleotide sequence (SEQ ID NO: 1) of the FIV Petaluma strain (GenBank Accession No. M25381);

FIG. 2 illustrates the upstream or downstream positioning of various primers relative to the consistent breakpoint identified within the FIV gag gene region;

FIG. 3 is an electrophoretic gel evidencing PCR amplification of FIV proviral DNA of various subtypes using an FIV gag-specific primer, Gag1-r, and an FIV-specific primer, LTR1-f;

FIG. 4 is an electrophoretic gel evidencing PCR amplification of FIV proviral NA of various subtypes using an FIV gag-specific primer, Gag 781-r, and an FIV-specific primer, Gag 635-f;

FIG. 5 illustrates the positioning of LTR1-f, LTR2-f, Gag1-r, Gag635f and Gag718r primers relative to the FIV Petaluma genome.

### DETAILED DESCRIPTION OF THE INVENTION

The present invention is directed to a method of diagnosing any feline immunodeficiency virus (FIV) infection in a cat through the use of a polymerase chain reaction (PCR) assay. DNA from a DNA-containing sample obtained from a cat to be diagnosed is subjected to PCR utilizing a first FIV gag-specific primer and a second FIV-specific primer to form an FIV-specific amplicon. The detection of an FIV-specific amplicon following the PCR amplification indicates FIV infection in the subject cat. The use of an FIV gag-specific primer advantageously allows for the exponential amplification of proviral DNA of any FIV subtype, thereby providing a universal means to diagnose FIV infection.

As used herein, the term “FIV” means feline immunodeficiency virus including all strains, such as the Petaluma strain and strain USgaB01; all subtypes, such as subtypes A through E, and evolving subtypes B such as FIV Texas (Weaver et al, 2004); isolates including isolates from Asia (Kakinuma et al. 1995), Europe (Pistello et al., 1997), Australia (Greene et al., 1993) and North America; and variants and recombinants thereof.

As used herein, the term “viral subtype” refers generally to FIV genomes that have greater than 80% similarity to each other and exhibit at least 20% divergence within the FIV DNA sequence of the env gene from other clusters, including for example FIV subtypes A through E.

The term “primer” as used herein is meant to encompass a single-stranded DNA fragment that is capable of binding to an FIV genomic template to initiate the formation of an amplicon in the PCR amplification process. Such PCR primers are typically about 10 to 35 nucleotide bases in length; however, primers outside of this size range may also be used as one of skill in the art will appreciate.

A primer “derived” from a particular FIV DNA sequence or region of the FIV genome, i.e. primer region, refers to the fact that the primer may have a forward or reverse complementary sequence to the DNA sequence on the strand of DNA upon which the primer binds, and may also vary from the native sequence by nucleotide substitutions, deletions or insertions which do not affect its function as a primer for use in PCR.

As used herein, the term “amplicon” refers to the DNA product that results from a polymerase chain reaction (PCR). The amplicon is defined by the primers which are designed to allow for the amplification of a particular DNA product. The amplicon, thus, is a DNA product which includes the primer regions, i.e. the regions to which each of the PCR primers bind, as well as the sequence between the primer regions.

To conduct the diagnostic method of the present invention, a DNA-containing biological sample is obtained from a cat to be diagnosed. Appropriate DNA-containing biological samples for use in the present method include, but are not limited to, saliva, urine, semen and other bodily secretions, as well as hair, epithelial cells and the like. Although such non-invasively obtained biological samples are preferred for use in the present method, one of skill in the art will appreciate that invasively-obtained DNA-containing biological samples, may also be used in the present method, including for example, blood, serum, bone marrow, cerebrospinal fluid (CSF) and tissue biopsies such as lymph node samples. Techniques for the invasive process of obtaining such samples are known to those of skill in the art.

It may be necessary, or preferable, to extract the DNA from the biological sample prior to proceeding with the PCR amplification. Methods of DNA extraction are well-known to those of skill in the art and include chemical extraction techniques utilizing phenol-chloroform (Sambrook et al., 1989), guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN (Chatsworth, Calif.), or the Extract-N-Amp blood kit available from Sigma (St. Louis, Mo.).

Once an appropriate DNA sample is obtained, it is subjected to PCR amplification using primers derived from FIV in order to determine whether or not FIV DNA is present in the sample, and thus, whether there is FIV infection in the subject cat. DNA amplification by PCR is a technique that is readily known by those of skill in the art, and described in U.S. Pat. No. 4,683,195. Generally, PCR allows for the amplification of a DNA template strand, the termini of which are defined by a forward and reverse primer pair, by the repeated polymerase-assisted synthesis of the DNA strand. PCR conditions may vary depending on the length and nucleotide sequence of the primer. For example, an annealing temperature of less than 50° C. may be required when one, or both, of the PCR primers is less than 20 nucleotides in length or contains a low percentage of guanine and/or cytosine bases. Preferably, an annealing temperature of 50 to 72° C., and more preferably 55 to 65° C. is used for PCR. As well, the concentration of MgCl₂ utilized in the PCR buffer may vary depending on the nucleotide base composition and length of the PCR primers. The concentration of MgCl₂ utilized may be in the range of 0.5 to 3.0 mM; however, a more preferable concentration range is that of 1.0 to 2.5 mM, and most preferable is a concentration range of 1.4 to 2.0 mM. The length of time allowed for the thermostable DNA polymerase to extend the daughter strand of DNA will vary depending on the nucleotide length of the amplicon. Generally, an extension time in the range of 45 to 120 seconds for every one thousand nucleotide bases of the amplicon is appropriate; however, an extension time of 50 to 100 seconds is preferred, and an extension time of 60 to 90 seconds is most preferred.

To diagnose FIV infection in a cat using the present PCR method, a first FIV gag-specific PCR primer is utilized. The term “gag-specific” as used herein is meant to denote a primer that is specific to the gag region of an FIV genome, for example, a primer that exhibits at least about 80% sequence homology with an FIV gag sequence and retains primer function. Preferably the primer exhibits at least about 90% sequence homology with an FIV gag sequence, and most preferably, the primer exhibits at least 95% sequence homology with an FIV gag sequence. The term “gag” refers to the gag gene region of an FIV genome which encodes structural capsid proteins. In the Petaluma FIV strain, for example, the gag region is defined by nucleotides 628 to 1980 of the genome. However, as will be appreciated by one of skill in the art, the exact nucleotide location of this region may vary somewhat among FIV strains and subtypes. For use in the present method, the gag-specific primer is derived from a region of the gag gene that is highly conserved among FIV, e.g. a region exhibiting at least 95% sequence homology among FIV. In one embodiment of the present invention, as described in more detail in the specific examples herein, the gag-specific primer is derived from the 5′ end of the gag gene, for example, the region spanning nucleotides 628 to 1000. Preferably, the gag-specific primer is derived from the gag nucleotide region 700 to 800, and more preferably, from the gag nucleotide region 768 to 788. In another embodiment, the gag-specific primer is derived from the central region of the gag gene, for example, the region spanning nucleotides 1200 to 1500. Preferably, the gag-specific primer is derived from the gag nucleotide region 1350 to 1450, and more preferably, from the gag nucleotide region 1409 to 1389. Moreover, the gag-specific primer is also defined by a GC content between about 45% to 60% and a melting temperature (Tₘ) between about 50° C. to 70° C. and possesses no variation in at least the three nucleotides at the 3′ end of the primer from the FIV gag gene sequence.

The second PCR primer of the required PCR primer pair used in the present method is an FIV-specific primer. The term “FIV-specific” as used herein is meant to denote a primer that is specific to FIV, i.e. exhibiting suitable sequence homology with a region in the FIV genomic template such that the primer readily binds to the FIV template under standard PCR conditions, for example a sequence homology of at least about 80% with a region in an FIV genome, and more preferably at least about 90% sequence homology with a region of the FIV genome, but is not derived from any particular region of the FIV genome. It is important, however, that the FIV-specific primer be suitable to function effectively as a second primer in the present PCR method, i.e. the primer binds to a region of an FIV genome suitable for PCR amplification to occur, for example to a region which is appropriately spaced from the binding region of the gag-specific primer to allow for amplification to occur. In this regard, it is preferable that the second primer be directed to a region that is within about 1500 nucleotides from the binding region of the FIV gag-specific primer. The FIV-specific primer may, for example, be derived from the 5′ LTR or long terminal repeat region of the FIV genome such as the region defined by nucleotides 1 to 355 inclusive of an FIV genome. Specific examples of suitable primers derived from this LTR region include, but are not limited to, primers derived from nucleotides 122-141 of the LTR and primers derived from nucleotides 285-309 of the LTR. The FIV-specific primer may also be derived from within the gag region of the FIV genome, within nucleotides 628 to 1980 of the FIV genome. Specific examples of suitable FIV-specific primers derived from the gag region include primers derived from nucleotides 625 to 643 of FIV gag. As set out above, the FIV-specific primer may be based on or derived from a native FIV sequence, but may include some nucleotide variation such as nucleotide insertion, deletion or substitution which does not affect its performance as a primer.

The FIV primers used in the present PCR method are produced using methods of DNA synthesis well known to those of skill in the art, including for example, the phosphotriester or phosphodiester methods described in U.S. Pat. No. 4,683,195, the contents of which are incorporated herein by reference, either manually or through the use of an automated DNA synthesizing apparatus, such as an ABI 3900 High-Throughput DNA Synthesizer as manufactured by Applied Biosystems.

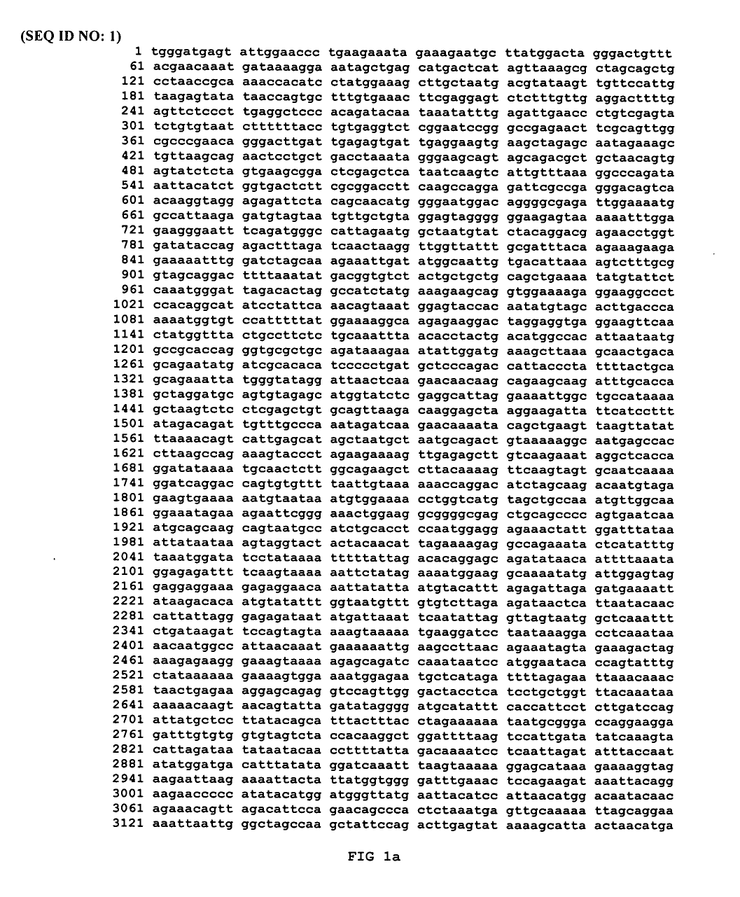

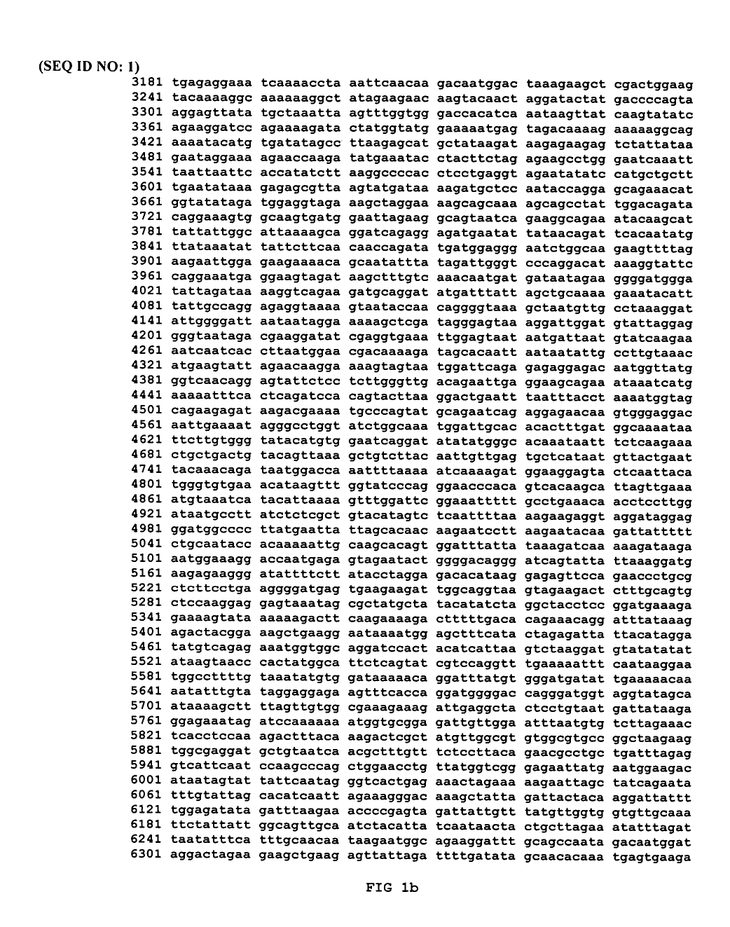

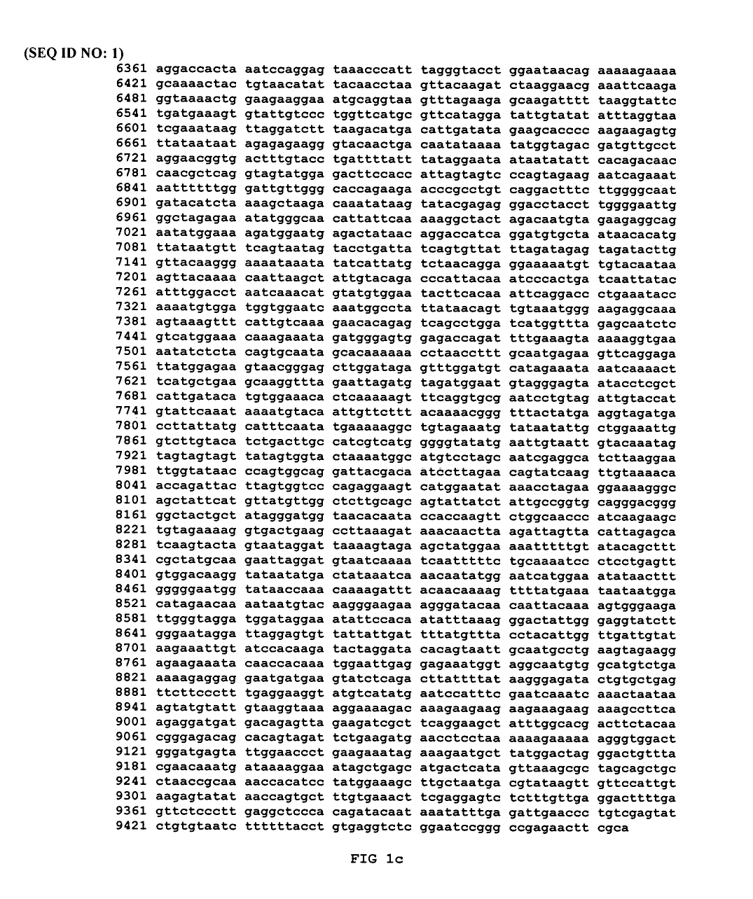

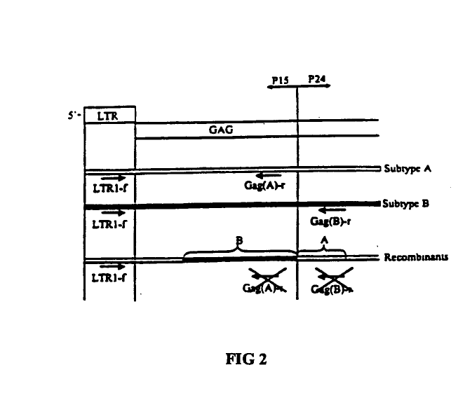

Given the availability of FIV nucleotide sequence information, as set out in FIG. 1 herein, FIV gag-specific and FIV-specific primers for use in the present PCR method may be designed. Publicly available sequence databases, such as GenBank (http://www.ncbi.nlm.nih.gov/), permit access to sequences of other FIV strains or subtypes. The conservation of such FIV genome sequences can readily be established through the use of various web-based sequence alignment tools, such as the CLUSTAL W Multiple Sequence Alignment (http://clustalw.genome.ad.jp/), as would be appreciated by one of skill in the art.

Once the PCR amplification is completed, it is necessary to determine whether or not a targeted FIV-specific amplicon has been produced. The term “targeted” refers to an FIV-specific amplicon that should have resulted from the PCR amplification using the selected gag-specific and FIV-specific primers if FIV DNA was present in the sample, i.e. if the cat from which the sample was obtained was infected by FIV. This can be accomplished by various methods that are known to those of skill in the art, such as by electrophoresis and Southern blot analysis (see Sambrook et al., 1989; Southern, 1975). For example, amplicon determination may be conducted by subjecting a portion of the PCR reaction product to analysis by electrophoresis against known DNA size markers. Southern blot analysis of the electrophoresed sample is then conducted to determine if the desired FIV-specific amplicon resulted from the PCR amplification process by hybridization to known DNA sequences.

A diagnosis of FIV is made when it is determined that the PCR produced the targeted FIV-specific amplicon. The production of the amplicon is a clear indication that FIV DNA is present in the DNA sample extracted from the cat, and thus, evidence of FIV infection. The absence of the targeted FIV-specific amplicon is a clear indication that FIV DNA is not present in the DNA sample, and thus, that there is no FIV infection. The present method is used to directly determine the presence of FIV DNA as opposed to methods which indirectly detect immunological products of FIV infection, e.g. antibodies. As a result, the present method advantageously precludes false positive results which readily occur in diagnostic methods which utilize antibody determination. For example, due to the availability of optional FIV vaccination, a false positive result can occur when immunological FIV diagnostic tests are used to diagnose vaccinated cats because the presence of FIV antibodies in a vaccinated cat is not necessarily indicative of FIV infection.

In one embodiment of the present invention, DNA extracted from whole blood samples obtained from FIV-infected cats were assayed using the present PCR method. As described in detail in the specific examples that follow, the FIV gag-specific primer used in the PCR was derived from nucleotides 1409 to 1389 (i.e. the reverse complementary sequence to nucleotides 1389 to 1409) of the gag region having the following nucleotide sequence: 5′-AGATACCATGCTCTACACTGC-3′ (SEQ ID No: 4). The FIV-specific primers used were derived from the LTR region, nucleotides 122-141 and 285-309, having the following specific sequences, respectively: 5′-TTA ACCGCAAAACCACATCC-3′ (SEQ ID No: 2) and 5′-TGAACCCTGTCGTGTATCTGTGTAA-3′ (SEQ ID No: 3). As set out above, and as one of skill in the art will appreciate, these primer sequences are exemplary only. Gag-specific primers which exhibit at least 80% sequence homology with SEQ ID No: 4 and are functionally equivalent, i.e. retain primer function, may also be used, as well as functionally equivalent primers derived from other nucleotide regions of gag which are highly conserved in all FIV. FIV-specific primers which exhibit at least about 80% sequence homology with either SEQ ID No: 2 or 3 and are functionally equivalent thereto, as well as FIV-specific primers derived from other nucleotide regions of the LTR and surrounding gene regions may similarly be used as long as the FIV-specific primer is derived from a region which is spaced suitably from the gag-specific primer in order to permit PCR to occur. Sequence modifications which may be made to the specific exemplified primers to yield functionally equivalent primers without significant loss of function include nucleotide base substitution, deletion and insertion, within an acceptable range of sequence modification. With respect to the gag-specific primer, functional equivalents include primers which exhibit at least about 80% sequence homology with a gag region that is highly conserved in all FIV, preferably 90% sequence homology and more preferably 95% sequence homology, while retaining a GC content of between about 45% to 60%, a melting temperature of between about 50° C. to 70° C., and having no variation in the four nucleotides at the 3′-most end of the primer from those in the native FIV gag sequence. Accordingly, examples of a functionally equivalent gag-specific primer include those which have up to 4 nucleotide substitutions within the 18 nucleotides situated proximal to the 5′ end of the primer identified as SEQ ID No: 4, which do not result in the formation of a structure that might inhibit the function of the primer (e.g. a hairpin structure), while retaining the GC content and melting temperature indicated above.

In another embodiment of the present invention, DNA extracted from whole blood samples obtained from FIV-infected cats were assayed using the present PCR method. As described in detail in the specific examples that follow, the FIV gag-specific primer used in the PCR was derived from nucleotides 788-768 (i.e. the reverse complementary sequence to nucleotides 768-788) of the gag region having the following nucleotide sequence: 5′-GGTATATCACCAGGTTCTGCT-3′ (SEQ ID No: 6). The FIV-specific primer used was also derived from the gag region, nucleotides 625-643, having the following specific sequence: 5′-AACATGGGGAATGGACAGG-3′ (SEQ ID No: 5).

In a further aspect of the present invention a kit is provided comprising a first FIV gag-specific primer which binds to a first region on an FIV genome and a second FIV-specific primer which binds to a second region on an FIV genome, and wherein the first and second regions are spaced such that amplification can occur. The kit may consist of a variety of primer combinations derived from any FIV genome that allow for the diagnosis of FIV infection in a cat. The first FIV gag-specific primer is derived from the FIV gag region, generally nucleotides 628-1980 in the FIV genome. The second FIV-specific primer is not particularly restricted with respect to the FIV gene region from which it is derived; however, as set out above, it is important that the FIV-specific primer be derived from a region of an FIV genome that is appropriately spaced from the binding region of the gag-specific primer to allow for amplification to occur.

In a further aspect of the present invention, an FIV gag-specific primer is provided. The FIV gag-specific primer may be selected from the group consisting of: a primer comprising the sequence 5′-AGATACCATGCTCTACAC TGC-3′ (SEQ ID No: 4), a primer comprising the sequence 5′-GGTATATCACCAGGTTCTGCT-3′ (SEQ ID No: 6) and functionally equivalent variant primers comprising a nucleotide sequence which exhibits at least about 95% sequence homology to one of SEQ ID No: 4 or SEQ ID No: 6. Functionally equivalent variants should retain a GC content of between about 45% to 60%, a melting temperature of between about 50° C. to 70° C., and have no variation in the three nucleotides at the 3′ end of the primer from those in the native FIV gag sequence. Functional variants preferably comprise at least the nucleotides -CACTGC- at the 3′ end of the primer.

Embodiments of the invention will now be described by reference to the following specific examples which are not to be construed as limiting.

### SPECIFIC EXAMPLES

### Example 1

### Proviral Amplification of FIV LTR-gag Region

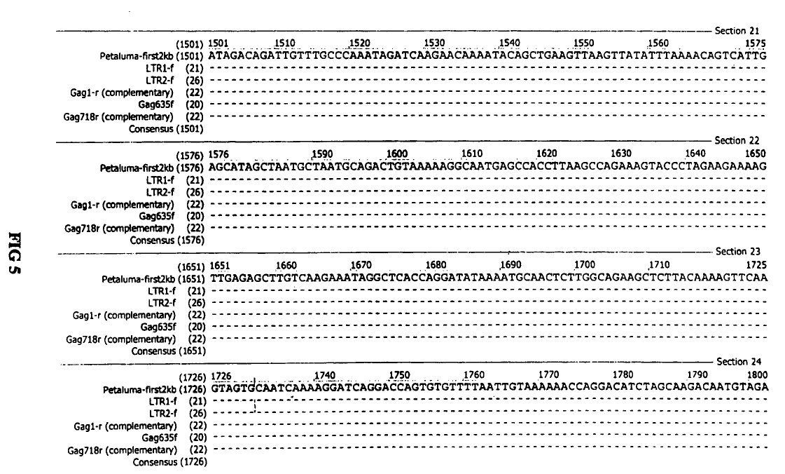

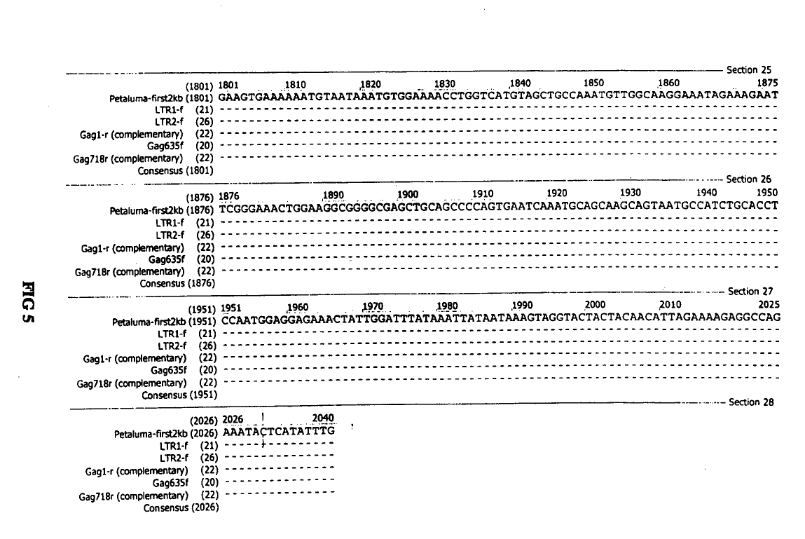

The amplification of proviral DNA from the LTR-gag gene region from four different subtypes of FIV was accomplished using a primer combination wherein the forward FIV-specific primer (LTR1-f, SEQ ID No: 2) was able to recognize a highly conserved sequence within the LTR region of FIV and the reverse gag-specific primer (Gag1-r, SEQ ID No: 4) was able to recognize a highly conserved sequence within the gag gene region. FIG. 5 illustrates the relative positioning of these primers relative to the FIV genome.

Materials. Reference FIV subtype A, strain Petaluma (clone 34TF10), was obtained from J. Elder, through the AIDS Research and Reference Reagent Program, Division of AIDS, NIAID, NIH. Subtype C was obtained from J. Mullins, University of Washington, Seattle, Wash.; and subtype D, strain Shizuoka, by T. Hohdatsu, Kitasato University, Japan. Of the 29 unknown samples, 4 originated from the Eastern United States, 1 from British Columbia, and 24 from Ontario. All feline blood samples that were from Canada and the United States are as set out in Table 1 below. The blood samples were confirmed positive for FIV antibodies by enzyme-linked immunosorbent assay (ELISA) (Pet Check ELISA; IDEXX, Portland, Me.) and yielded an FIV-specific amplicon through PCR amplification. Background information on the samples is provided in Table 1. Base pair identification of all sequences including primers and amplicons is in reference to FIV subtype A, strain Petaluma (GenBank Accession No. M25381) (Talbott et al., 1989).

TABLE 1
Sample description and subtype distribution of 29 FN primary isolates^{a}

| | Province City/ | | | | | |
|---|---|---|---|---|---|---|
| Sample Country | State | | Age | Sex | Clinical findings | Subtype |
| CaONA01 Canada | ON | Mount Hope | 10 | FN | Anemia, cutaneous ulceration | A |
| CaONA02 Canada | ON | Churchill | 8 | MN | Hyperglobuhnemia, lymphopenta, cardiomyopathy | A |
| CaONA03 Canada | ON | Guelph | 4 | M | Proliferative polyarthritis, lymphopenia | A |
| CaONA04 Canada | ON | Scarborough | 5 | FN | Severe stomatitis | A |
| CaONA05 Canada | ON | Oakville | 8 | MN | Severe stomatitis, diabetes mellitus | A |
| CaONA06 Canada | ON | Kincardine | 9 | MN | Demodecosis, hyperglobulinemia, lymphopenia | A |
| CaONA07 Canada | ON | Guelph | 10 | MN | Stomatitis, diarrhea | A |
| CaONA08 Canada | ON | Toronto | 6 | MN | Anaplastic extranodal lymphoma | A |
| CaONA09 Canada | ON | Guelph | 9 | MN | Chronic diarrhea, stomantis | A |
| CaONA10 Canada | ON | Guelph | NA | MN | Pneumonia, cutaneous ulceration, lymphopenia | A |
| CaONA11 Canada | ON | Guelph | 7 | MN | Stomatitis, diarrhea | A |
| CaONA12 Canada | ON | Toronto | 2 | MN | Stomatitis, neutropenia | A |
| CaONA13 Canada | ON | Toronto | 4 | M | Vomiting, cutaneous ulceration | A |
| CaONA14 Canada | ON | Toronto | NA | F | Clinically healthy | A |
| CaONA15 Canada | ON | Toronto | NA | NA | Gingivitis, diarrhea, multiple cutaneous abscesses | A |
| CaONA16 Canada | ON | Toronto | 2 | M | NA | A |
| CaONA17 Canada | ON | Ottawa | 6 | MN | Diarrhea, neutropenia | A |
| CaONAB01 Canada | ON | Toronto | 5 | FN | Chronic renal disease | A/B |
| CaONAB02 Canada | ON | Brampton | 5 | MN | Multiple cutaneous abscesses | A/B |
| CaCINAB03 Canada | ON | Kitchener | NA | NA | Anemia, lymphocytosis | A/B |
| CaONB01 Canada | ON | Guelph | 7 | MN | Uveitis | B |
| CaONB02 Canada | ON | Guelph | 10 | F | Encephalopathy, hyperglobulinemia | B |
| CaONB03 Canada | ON | Barrie | 6 | MN | Gingivitis, abscessation, diarrhea | B |
| CaONB04 Canada | ON | Toronto | NA | NA | NA | B |
| USctB01 USA | CT | Plainville | NA | M | Gingivitis, diarrhea | B |
| USgaB01 USA | GA | Athens | 8 | MN | Seizures, lymphopenia | B |
| USgaB02 USA | GA | Athens | 8 | MN | Stomatitis, diarrhea | B |
| USgaB03 USA | GA | Athens | 5 | MN | Lymphadenopathy | B |
| CaBC01 Canada | BC | Vancouver | 8 | MN | Stomatitis | C |

^{a}Abbreviations: ON, Ontario; FN, female neutered; MN, male neutered; CT, Connecticut; GA, Georgia; BC, British Columbia; NA, not available.

DNA isolation. DNA was extracted from 200 μl of each whole blood sample using the QIAamp DNA Blood Minikit (QIAGEN, Chatsworth, Calif.) following the directions of the manufacturer. The DNA was eluted into 100 μl of buffer and stored at −20° C.

LTR-gag PCR. Primers were designed to amplify the LTR-gag region of proviral DNA. First round reactions produced amplicons of 1287 bp using primers LTR1-f (SEQ ID No: 2) and Gag1-r (SEQ ID No: 4) (Table 2). Utilization of a different forward primer in the PCR, namely LTR2-f (SEQ ID No:3), along with the Gag1-r primer (SEQ ID No: 4), resulted in an FIV-specific amplicon of 1127 bp. The primer sequences are set out in Table 2 below. This DNA fragment included the LTR regions R and U5, the gag region coding for the p15 protein and 421 bp of the gag region coding for the p24 protein. Amplifications were performed in 25 μl total volume with 1.25 units of Taq DNA polymerase (Life Technologies, Burlington, ON, Canada), 1.5 mM MgCl₂, 0.2 mM of each dNTP, 0.5 μM of each primer and 2 μl of purified DNA. If an insufficient amount of an FIV-specific amplicon was generated during an initial PCR reaction, 2 μl of the particular initial PCR reaction product was used as a template for a subsequent PCR re-amplification treatment. Reactions were cycled 35 times with denaturation at 94° C. for 45 sec, annealing at 60° C. for 20 sec, and elongation at 72° C. for 90 sec. Conditions for a PCR re-amplification treatment were identical as for the initial PCR amplification except the annealing temperature was 64° C. for 20 sec.

TABLE 2
Primer Sequence and Location.

| | | | |
|---|---|---|---|
| SEQ ID No. | Primer | Sequence (5′ → 3′) | Location (bp)^{a} |

| | | | |
|---|---|---|---|
| SEQ ID No: 2 | LTR1-f | TTAACCGCAAAACCACATCC | 122-141 |
| SEQ ID No: 3 | LTR2-f | TGAACCCTGTCGTGTATCTGTGTAA | 285-309 |
| SEQ ID No: 4 | Gag1-r | AGATACCATGCTCTACACTGC | 1409-1389 |

^{a}All positions are in reference to subtype A Petaluma (GenBank Accession No. M25381)

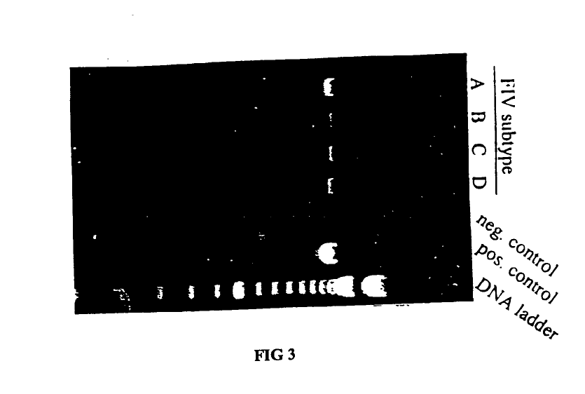

Results. PCR products were identified by electrophoresis in 1% agarose gels and ethidium bromide staining. The results confirm that the present primers, designed to encompass the LTR-gag region, consistently amplified proviral DNA of subtypes A, B, C and D. Results using the Gag1-r and LTR1-f primers are illustrated in FIG. 3 in which there is evidence that the 1287 bp amplicon is produced. Reference strains Petaluma (A), FIV-C (C) and Shizuoka (D), as well as 27 of the 29 primary isolates were efficiently amplified after an initial round of PCR; while 2 of 29 samples required a PCR re-amplification treatment in order to allow for amplicon detection. These results demonstrate that this was a highly efficient amplification protocol for detection of proviral FIV DNA. Although subtype E DNA samples were not available, LTR1-f (SEQ ID No: 2) and Gag1-r (SEQ ID No: 4) primers were 100% homologous to corresponding regions in the 5 known FIV subtypes, including subtype E, and, thus, are expected to amplify proviral DNA of subtype E as well.

Sample preparation for sequencing. Amplicons of the appropriate size were cut out of agarose gels with sterile blades, and the DNA was extracted from the agarose gel using a QIAquick Gel Extraction kit (QIAGEN, Chatsworth, Calif.). The concentration of purified DNA samples was determined by fluorimetry (PicoGreen, Roche, Montreal, PQ) and adjusted to 30 ng/μl. Sequencing primers were the same as for PCR amplification. Amplicon sequences were determined/confirmed by the BigDye Terminator method on an ABI Prism 377 XL DNA Sequencer (DNA Sequencing Facility, Robarts Research Institute, London, ON).

### Example 2

### Proviral Amplification of FIV gag Region

Amplification of proviral DNA from the gag gene region FIV subtypes A, B, C and D (as identified in Example 2) was accomplished using a primer combination in which both the forward and reverse primers were able to recognize conserved sequences within the gag region of the FIV genome. The forward FIV-specific primer (Gag 635 f) had a nucleotide sequence as follows: AACATGGGGAATGGACAGG (SEQ ID No: 5) and was derived from nucleotide positions 625 to 643 of the gag gene. This represents nucleotide positions 625 to 643 of the FIV genome as shown in FIG. 5. The reverse FIV gag-specific primer (Gag 781 r) had the nucleotide sequence: GGTATATCACCAGGTTCTGCT (SEQ ID No: 6) and was derived from nucleotide positions 788 to 768 of the gag gene. This represent nucleotide positions 768 to 788 of the FIV genome as shown in FIG. 5.

First round PCR reactions using these gag primers produced amplicons of 146 bp using primers. Amplifications were performed in 25 μl total volume with 1.25 units of Taq DNA polymerase (Life Technologies, Burlington, ON, Canada), 1.5 mM MgCl₂, 0.2 mM of each dNTP, 0.5 μM of each primer and 2 μl of purified DNA. If an insufficient amount of an FIV-specific amplicon was generated during an initial PCR reaction, 2 μl of the particular initial PCR reaction product was used as a template for a subsequent PCR re-amplification treatment under the same conditions. Reactions were cycled 35 times with denaturation at 94° C. for 30 sec, annealing at 60° C. for 30 sec, and elongation at 72° C. for 30 sec.

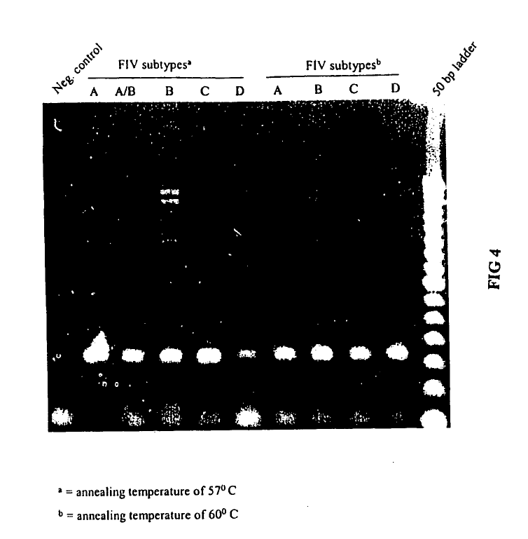

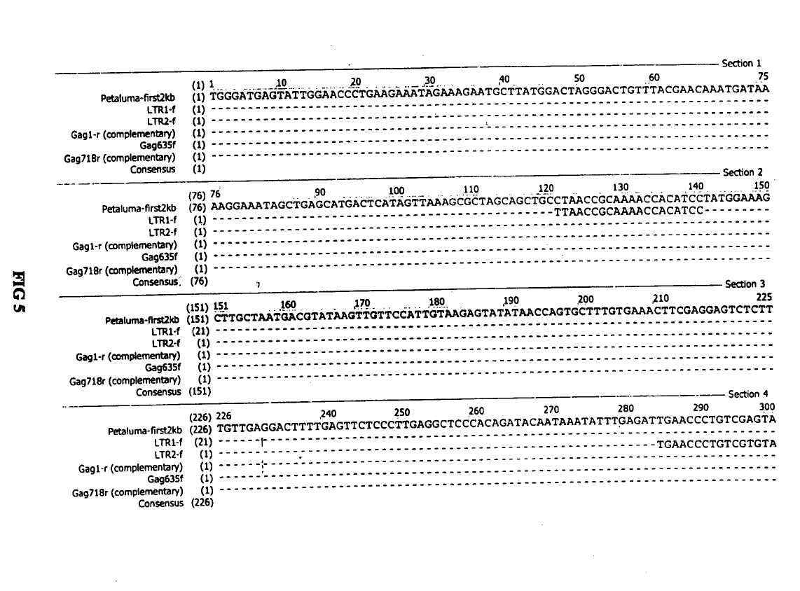

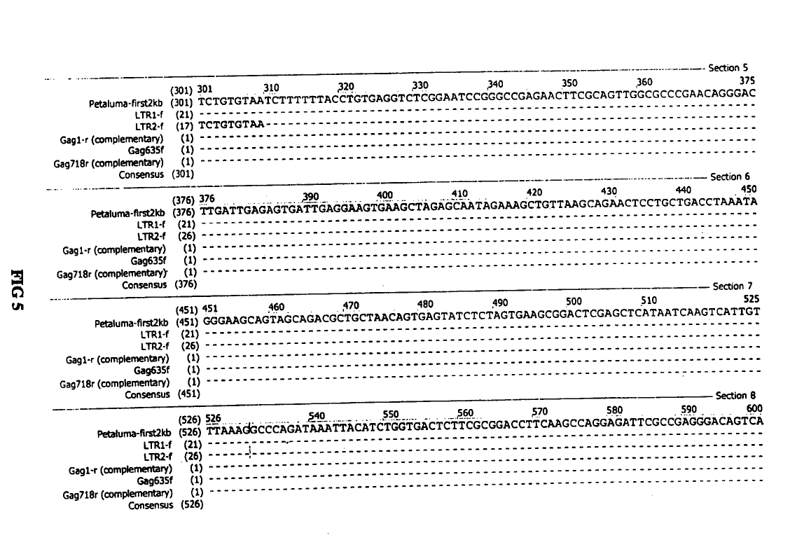

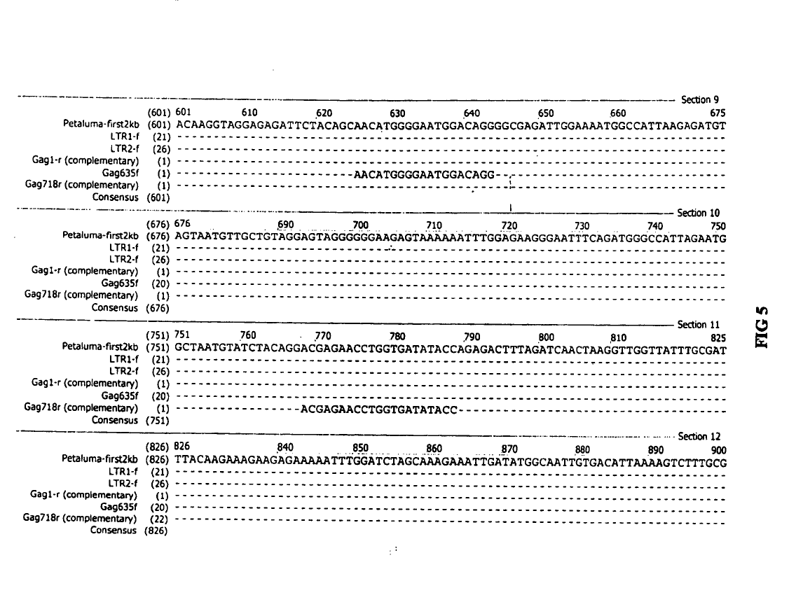

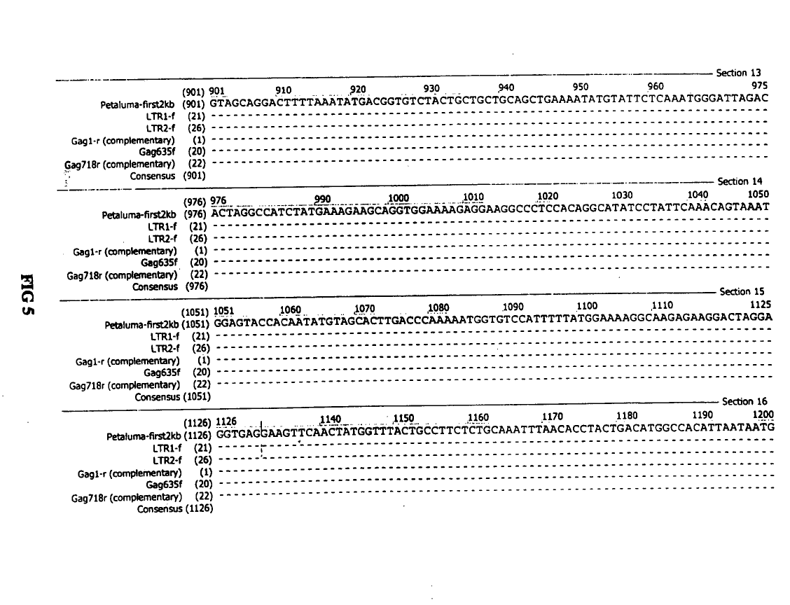

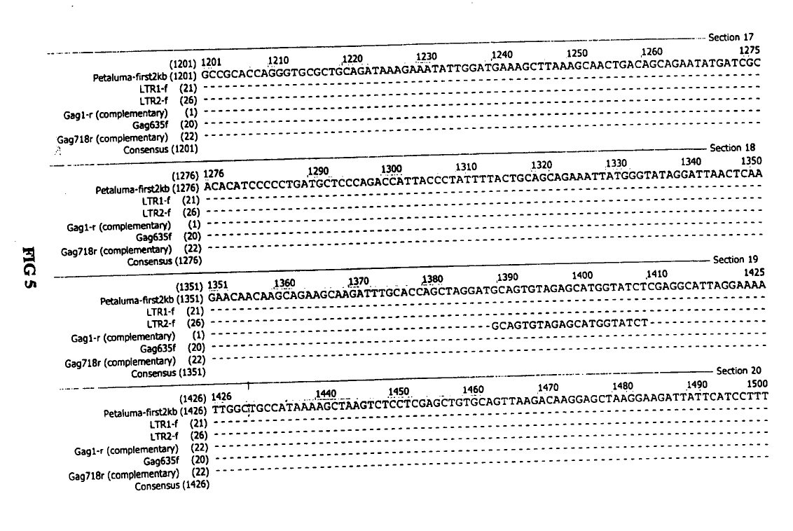

Results. PCR products were identified by electrophoresis in 1% agarose gels and ethidium bromide staining. Results from these proviral amplification experiments are illustrated in FIG. 4 and confirm that gag-specific primers consistently amplified proviral DNA of subtypes A, B, C and D. These results demonstrate that this was a highly efficient amplification protocol for universal detection of proviral FIV DNA.

### REFERENCES

U.S. Patent Documents

Tompkins et al . . . U.S. Pat. No. 5,478,724.
Mullis et al . . . U.S. Pat. No. 4,683,195.
   Other Publications
Bachmann, M. H., C. Mathiason-Dubard, G. H. Learn, A. G. Rodrigo, D. L. Sodora, P. Mazzetti, E. A. Hoover, and J. I. Mullins. 1997. Genetic diversity of feline immunodeficiency virus: dual infection, recombination, and distinct evolutionary rates among envelope sequence clades. J Virol 71: 4241-53.
Bendinelli, M., M. Pistello, S. Lombardi, A. Poli, C. Garzelli, D. Matteucci, L. Ceccherini-Nelli, G. Malvaldi, and F. Tozzini. 1995. Feline immunodeficiency virus: an interesting model for AIDS studies and an important cat pathogen. Clin Microbiol Rev 8:8713 112.
Connell, S. 2003. Manufacturer addresses concerns about FIV vaccine. J Am Vet Med Assoc 222: 149.
Gao, F., D. L. Robertson, C. D. Carruthers, S. G. Morrison, B. Jian, Y. Chen, F. Barre Sinoussi, M. Girard, A. Srinivasan, A. G. Abimiku, G. M. Shaw, P. M. Sharp, and B. H. Hahn. 1998. A comprehensive panel of near-full-length clones and reference sequences for non-subtype B isolates of human immunodeficiency virus type 1. J Virol 72:5680-98.
Greene, W. K., J. Meers, G. del Fierro, P. R. Carnegie, and Robinson, W. F. 1993. Nucleotide sequence of Australian isolates of the feline immunodeficiency virus: a comparison with other feline lentiviruses. Arch Virol 132: 369-379.
Hohdatsu, T., H. Hirabayashi, K. Motokawa, and H. Koyama. 1996. Comparative study of the cell tropism of feline immunodeficiency virus isolates of subtypes A, B and D classified on the basis of the env gene V3-V5 sequence. J Gen Virol 77 (Pt 1):93-100.
Hohdatsu, T., K. Motokawa, M. Usami, M. Amioka, S. Okada, and H. Koyama. 1998. Genetic subtyping and epidemiological study of feline immunodeficiency virus by nested polymerase chain reaction-restriction fragment length polymorphism analysis of the gag gene. J Virol Methods 70:107-11.
Ishida, T., T. Washizu, K. Toriyabe, S. Motoyoshi, I. Tomoda, and N. C. Pedersen. 1989. Feline immunodeficiency virus infection in cats of Japan. J Am Vet Med Assoc 194:2214 5.
Johnson, C. M., B. A. Torres, H. Koyama, and J. K. Yamamoto. 1994. Tenth anniversary perspectives on AIDS. FIV as a model for AIDS vaccination. AIDS Res Hum Retroviruses 10:225-8.
Jost, S., M. C. Bernard, L. Kaiser, S. Yerly, B. Hirschel, A. Samri, B. Autran, L. E. Goh, and L. Perrin. 2002. A patient with HIV-1 superinfection. N Engl J Med 347:731-6.
Kakinuma, S., K. Motokawa, T. Hohdatsu, J. K. Yamamoto, H. Koyama, and H. Hashimoto. 1995. Nucleotide sequence of feline immunodeficiency virus: classification of Japanese isolates into two subtypes which are distinct from non-Japanese subtypes. J Virol 15 69:3639-46.
Kyaw-Tanner, M. T., W. K. Greene, H. S. Park, and W. F. Robinson. 1994. The induction of in vivo superinfection and recombination using feline immunodeficiency virus as the model. Arch Virol 138:261-71.
Langley, R. J., V. M. Hirsch, S. J. O'Brien, D. Adger Johnson, R. M. Goeken, and R. A. Olmsted. 1994. Nucleotide sequence analysis of puma lentivirus (PLV-14): genomicorganization and relationship to other lentiviruses. Virology 202:853-64.
Lole, K. S., R. C. Bollinger, R. S. Paranjape, D. Gadkari, S. S. Kulkarni, N. G. Novak, R. Ingersoll, H. W. Sheppard, and S. C. Ray. 1999. Full-length human immunodeficiency virus type 1 genomes from subtype C-infected seroconverters in India, with evidence of intersubtype recombination. J Virol 73:152-60.
Nishimura, Y., Y. Goto, H. Pang, Y. Endo, T. Mizuno, Y. Momoi, T. Watari, H. Tsujimoto, and A. Hasegawa. 1998. Genetic heterogeneity of env gene of feline immunodeficiency virus obtained from multiple districts in Japan. Virus Res 57:101-12.
Okada, S., R. Pu, E. Young, W. V. Stoffs, and J. K. Yamamoto. 1994. Superinfection of cats with feline immunodeficiency virus subtypes A and B. AIDS Res Hum Retroviruses 10:1739-46.
Olmsted, R. A., A. K. Barnes, J. K. Yamamoto, V. M. Hirsch, R. H. Purcell, and P. R. Johnson. 1989a. Molecular cloning of feline immunodeficiency virus. Proc Natl Acad Sci USA 86:2448-52.
Olmsted, R. A., V. M. Hirsch, R. H. Purcell, and P. R. Johnson. 1989b. Nucleotide sequence analysis of feline immunodeficiency virus: genome organization and relationship to other lentiviruses. Proc Natl Acad Sci USA 86:8088-92.
Pecoraro, M. R., K. Tomonaga, T. Miyazawa, Y. Kawaguchi, S. Sugita, Y. Tohya, C. Kai, M. E. Etcheverrigaray, and T. Mikami. 1996. Genetic diversity of Argentine isolates of feline immunodeficiency virus. J Gen Virol 77 (Pt 9):2031-5.
Pedersen, N. C., E. W. Ho, M. L. Brown, and J. K. Yamamoto. 1987. Isolation of a T-lymphotropic virus from domestic cats with an immunodeficiency-like syndrome. Science 235:790-3.
Pistello, M., G. Cammarota, E. Nicoletti, D. Matteucci, M. Curcio, D. Del Mauro, and M. Bendinelli. 1997. Analysis of the genetic diversity and phylogenedc relationship of Italian isolates of feline immunodeficiency virus indicates a high prevalence and heterogeneity of subtype B. J Gen Virol 78 (Pt 9):2247-57.
Sambrook, J., Fritsch, E. F. and T. Maniatis. 1989. Molecular Cloning: A Laboratory Manual. 2ed. (New York: Cold Spring Harbor Laboratory).
Siebelink, K. H., I. H. Chu, G. F. Rimmelzwaan, K. Weijer, A. D. Osterhaus, and M. L. Bosch. 1992. Isolation and partial characterization of infectious molecular clones of feline immunodeficiency virus obtained directly from bone marrow DNA of a naturally infected cat. J Virol 66:1091-7.
Siepel, A. C., A. L. Halpern, C. Macken, and B. T. Korber. 1995. A computer program designed to screen rapidly for HIV type 1 intersubtype recombinant sequences. AIDS Res Hum Retroviruses 11: 1413-6.
Sodora, D. L., E. G. Shpaer, B. E. Kitchell, S. W. Dow, E. A. Hoover, and J. L. Mullins. 1994. Identification of three feline immunodeficiency virus (FIV) env gene subtypes and comparison of the FIV and human immunodeficiency virus type 1 evolutionary patterns. J Virol 68:2230-8.
Southern, E. M. 1975. Detection of specific sequences among DNA fragments separated by gel electrophoresis. J Mol Biol 98: 503-517.
Sparkes, A. H., C. D. Hopper, W. G. Millard, T. J. Gruffydd Jones, and D. A. Harbour. 1993. Feline immunodeficiency virus infection. Clinicopathologic findings in 90 naturally occurring cases. J Vet Intern Med 7:85-90.
Talbott, R. L., E. E. Sparger, K. M. Lovelace, W. M. Fitch, N. C. Pedersen, P. A. Luciw, and J. H. Elder. 1989. Nucleotide sequence and genomic organization of feline immunodeficiency virus. Proc Natl Acad Sci USA 86:5743-7.
Weaver, E. A., Collisson, E. W., Slater, M. and Zhu, G. 2004. Phylogenetic analyses of Texas isolates indicate an evolving subtype of the Clade B feline immunodeficiency viruses. J Virol 78:2158-2163.
Worobey, M. and E. C. Holmes. 1999. Evolutionary aspects of recombination in RNA viruses. J Gen Virol 80 (Pt 10):2535-43.
Yamamoto, J. K., H. Hansen, E. W. Ho, T. Y. Morishita, T. Okuda, T. R. Sawa, R. M. Nakamura, and N. C. Pedersen. 1989. Epidemiologic and clinical aspects of feline immunodeficiency virus infection in cats from the continental United States and Canada and possible mode of transmission. J Am Vet Med Assoc 194:213-20.

All references referred to herein are hereby incorporated by reference.

1. A method for diagnosing FIV infection in a cat comprising the steps of:
a. obtaining a DNA-containing sample from the cat,
b. extracting the DNA from the sample,
c. subjecting the extracted DNA to PCR amplification to form an FIV-specific amplicon, wherein the amplification utilizes a first gag-specific primer which binds to a first region on an FIV genome and a second FIV-specific primer which binds to a second region on an FIV genome, and wherein the first and second regions are spaced such that amplification can occur; and
d. determining whether an FIV-specific amplicon is generated during step (iii), wherein the presence of an FIV-specific amplicon is indicative of an FIV infection in a cat.

2. A method as claimed in claim 1, wherein the second primer binds to a region in an FIV genome within no more than about 1500 nucleotides of the first primer.

3. A method as claimed in claim 1, wherein the FIV gag-specific primer is derived from nucleotides 1389-1409 of an FIV genome.

4. A method as claimed in claim 1, wherein the first primer comprises a sequence selected from the group consisting of: 5′-AGATACCATGCTCTACACTGC-3′ (SEQ ID No: 4) and functional equivalent sequences thereof which are at least 80% homologous thereto.

5. A method as claimed in claim 1, wherein the first primer has a GC content between about 45% to 60%, a melting temperature (Tₘ) between about 50° C. to 70° C. and possesses no variation in at least the four nucleotides at the 3′-most end of the primer identified in SEQ ID No: 4.

6. A method as claimed in claim 1, wherein the first primer comprises the sequence: 5′-AGATACCATGCTCTACACTGC-3′ (SEQ ID No: 4).

7. A method as claimed in claim 1, wherein the second primer is derived from the long terminal repeat (LTR) region of the FIV genome.

8. A method as claimed in claim 7, wherein the second primer is selected from the group consisting of: a primer comprising the sequence 5′-TTAACCGCAAAACCACATCC-3′ (SEQ ID No: 2); a primer comprising the sequence 5′-TGAACCCTGTCGTGTATCTGTGTAA-3′ (SEQ ID No: 3) and a functionally equivalent primer comprising a sequence which is at least 80% homologous with one of SEQ ID No: 2 and SEQ ID No:3.

9. A method as defined in claim 8, wherein the second primer is selected from the group consisting of: a primer comprising the sequence 5′-TTAACCGCAAAACCACATCC-3′ (SEQ ID No: 2) and a primer comprising the sequence 5′-TGAACCCTGTCGTGTATCTGTGTAA-3′ (SEQ ID No: 3).

10. A method as defined in claim 1, wherein the first and second primers are derived from the gag region of the FIV genome.

11. A method as defined in claim 1, wherein the second primer comprises a nucleotide sequence selected from the group consisting of: 5′-AACATGGGGAATGGACAGG-3′ (SEQ ID No: 5) and a nucleotide sequence which exhibits at least about 80% sequence homology thereto.

12. A method as defined in claim 1, wherein the first primer comprises a nucleotide sequence selected from the group consisting of: 5′-GGTATATCACCAGGTTCTCGT-3′ (SEQ ID No: 6) and a nucleotide sequence which exhibits at least about 80% sequence homology thereto.

13. A kit for diagnosing FIV infection in cats comprising a first FIV gag-specific primer which binds to a first region on an FIV genome and a second FIV-specific primer which binds to a second region on an FIV genome, and wherein the first and second regions are spaced such that amplification can occur.

14. A kit as claimed in claim 13, wherein the first FIV gag-specific primer is derived from nucleotides 1389-1409 of an FIV genome.

15. A kit as claimed in claim 13, wherein the first FIV gag-specific primer comprises a sequence selected from the group consisting of: a primer comprising the sequence 5′-AGATACCATGCTCTACAC TGC-3′ (SEQ ID No: 4) and a functionally equivalent primer comprising a nucleotide sequence which exhibits at least about 80% sequence homology with SEQ ID No: 4.

16. A kit as claimed in claim 13, wherein the second FIV-specific primer is selected from the group consisting of:
a primer comprising a nucleotide sequence 5′-TTAACCGCAAAACCACATCC-3′ (SEQ ID No: 2), a primer comprising a nucleotide sequence 5′-TGAACCCTGTCGTGTATCTGTGTAA-3′ (SEQ ID No: 3), a primer comprising a nucleotide sequence AACATGGGGAATGGACAGG-3′ (SEQ ID No: 5) and a functionally equivalent variant thereof comprising a nucleotide sequence which exhibits at least about 80% sequence homology to any one of SEQ ID No: 2, SEQ ID No:3 and SEQ ID No: 5.

17. A kit as claimed in claim 13, wherein the first FIV gag-specific primer comprises the sequence 5′-AGATACCATGCTCTACAC TGC-3′ (SEQ ID No: 4).

18. A kit as claimed in claim 17, wherein the second FIV-specific primer comprises a sequence selected from the group consisting of 5′-TTAACCGCAAAACCACATCC-3′ (SEQ ID No: 2) and 5′-TGAACCCTGTCGTGTATCTGTGTAA-3′ (SEQ ID No: 3).

19. An FIV gag-specific primer selected from the group consisting of: a primer comprising the sequence 5′-AGATACCATGCTCTACACTGC-3′ (SEQ ID No: 4) and a primer comprising the sequence GGTATATCACCAGGTTCTGCT (SEQ ID No: 6).

20. An FIV-specific primer selected from the group consisting of: a primer comprising a nucleotide sequence 5′-TTAACCGCAAAACCACATCC-3′ (SEQ ID No: 2), a primer comprising a nucleotide sequence 5′-TGAACCCTGTCGTGTATCTGTGTAA-3′ (SEQ ID No: 3) and a primer comprising a nucleotide sequence AACATGGGGAATGGACAGG-3′ (SEQ ID No: 5).


SEQUENCE LISTING (6 sequences)

SEQ ID NO: 1 - DNA, 9474 bp, Feline immunodeficiency virus
tgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattcgggaaactggaaggcggggcgagctgcagccccagtgaatcaaatgcagcaagcagtaatgccatctgcacctccaatggaggagaaactattggatttataaattataataaagtaggtactactacaacattagaaaagaggccagaaatactcatatttgtaaatggatatcctataaaatttttattagacacaggagcagatataacaattttaaataggagagattttcaagtaaaaaattctatagaaaatggaaggcaaaatatgattggagtaggaggaggaaagagaggaacaaattatattaatgtacatttagagattagagatgaaaattataagacacaatgtatatttggtaatgtttgtgtcttagaagataactcattaatacaaccattattagggagagataatatgattaaattcaatattaggttagtaatggctcaaatttctgataagattccagtagtaaaagtaaaaatgaaggatcctaataaaggacctcaaataaaacaatggccattaacaaatgaaaaaattgaagccttaacagaaatagtagaaagactagaaagagaagggaaagtaaaaagagcagatccaaataatccatggaatacaccagtatttgctataaaaaagaaaagtggaaaatggagaatgctcatagattttagagaattaaacaaactaactgagaaaggagcagaggtccagttgggactacctcatcctgctggtttacaaataaaaaaacaagtaacagtattagatataggggatgcatatttcaccattcctcttgatccagattatgctccttatacagcatttactttacctagaaaaaataatgcgggaccaggaaggagatttgtgtggtgtagtctaccacaaggctggattttaagtccattgatatatcaaagtacattagataatataatacaaccttttattagacaaaatcctcaattagatatttaccaatatatggatgacatttatataggatcaaatttaagtaaaaaggagcataaagaaaaggtagaagaattaagaaaattactattatggtggggatttgaaactccagaagataaattacaggaagaacccccatatacatggatgggttatgaattacatccattaacatggacaatacaacagaaacagttagacattccagaacagcccactctaaatgagttgcaaaaattagcaggaaaaattaattgggctagccaagctattccagacttgagtataaaagcattaactaacatgatgagaggaaatcaaaacctaaattcaacaagacaatggactaaagaagctcgactggaagtacaaaaggcaaaaaaggctatagaagaacaagtacaactaggatactatgaccccagtaaggagttatatgctaaattaagtttggtgggaccacatcaaataagttatcaagtatatcagaaggatccagaaaagatactatggtatggaaaaatgagtagacaaaagaaaaaggcagaaaatacatgtgatatagccttaagagcatgctataagataagagaagagtctattataagaataggaaaagaaccaagatatgaaatacctacttctagagaagcctgggaatcaaatttaattaattcaccatatcttaaggccccacctcctgaggtagaatatatccatgctgctttgaatataaagagagcgttaagtatgataaaagatgctccaataccaggagcagaaacatggtatatagatggaggtagaaagctaggaaaagcagcaaaagcagcctattggacagatacaggaaagtggcaagtgatggaattagaaggcagtaatcagaaggcagaaatacaagcattattattggcattaaaagcaggatcagaggagatgaatattataacagattcacaatatgttataaatattattcttcaacaaccagatatgatggagggaatctggcaagaagttttagaagaattggagaagaaaacagcaatatttatagattgggtcccaggacataaaggtattccaggaaatgaggaagtagataagctttgtcaaacaatgatgataatagaaggggatgggatattagataaaaggtcagaagatgcaggatatgatttattagctgcaaaagaaatacatttattgccaggagaggtaaaagtaataccaacaggggtaaagctaatgttgcctaaaggatattggggattaataataggaaaaagctcgatagggagtaaaggattggatgtattaggaggggtaatagacgaaggatatcgaggtgaaattggagtaataatgattaatgtatcaagaaaatcaatcaccttaatggaacgacaaaagatagcacaattaataatattgccttgtaaacatgaagtattagaacaaggaaaagtagtaatggattcagagagaggagacaatggttatgggtcaacaggagtattctcctcttgggttgacagaattgaggaagcagaaataaatcatgaaaaatttcactcagatccacagtacttaaggactgaatttaatttacctaaaatggtagcagaagagataagacgaaaatgcccagtatgcagaatcagaggagaacaagtgggaggacaattgaaaatagggcctggtatctggcaaatggattgcacacactttgatggcaaaataattcttgtgggtatacatgtggaatcaggatatatatgggcacaaataatttctcaagaaactgctgactgtacagttaaagctgtcttacaattgttgagtgctcataatgttactgaattacaaacagataatggaccaaattttaaaaatcaaaagatggaaggagtactcaattacatgggtgtgaaacataagtttggtatcccagggaacccacagtcacaagcattagttgaaaatgtaaatcatacattaaaagtttggattcggaaatttttgcctgaaacaacctccttggataatgccttatctctcgctgtacatagtctcaattttaaaagaagaggtaggataggagggatggccccttatgaattattagcacaacaagaatccttaagaatacaagattatttttctgcaataccacaaaaattgcaagcacagtggatttattataaagatcaaaaagataagaaatggaaaggaccaatgagagtagaatactggggacagggatcagtattattaaaggatgaagagaagggatattttcttatacctaggagacacataaggagagttccagaaccctgcgctcttcctgaaggggatgagtgaagaagattggcaggtaagtagaagactctttgcagtgctccaaggaggagtaaatagcgctatgctatacatatctaggctacctccggatgaaagagaaaagtataaaaaagacttcaagaaaagactttttgacacagaaacaggatttataaagagactacggaaagctgaaggaataaaatggagctttcatactagagattattacataggatatgtcagagaaatggtggcaggatccactacatcattaagtctaaggatgtatatatatataagtaacccactatggcattctcagtatcgtccaggtttgaaaaatttcaataaggaatggccttttgtaaatatgtggataaaaacaggatttatgtgggatgatattgaaaaacaaaatatttgtataggaggagaagtttcaccaggatggggaccagggatggtaggtatagcaataaaagcttttagttgtggcgaaagaaagattgaggctactcctgtaatgattataagaggagaaatagatccaaaaaaatggtgcggagattgttggaatttaatgtgtcttagaaactcacctccaaagactttacaaagactcgctatgttggcgtgtggcgtgccggctaagaagtggcgaggatgctgtaatcaacgctttgtttctccttacagaacgcctgctgatttagaggtcattcaatccaagcccagctggaacctgttatggtcgggagaattatgaatggaagacataatagtattattcaatagggtcactgagaaactagaaaaagaattagctatcagaatatttgtattagcacatcaattagaaagggacaaagctattagattactacaaggattattttggagatatagatttaagaaaccccgagtagattattgtttatgttggtggtgttgcaaattctattattggcagttgcaatctacattatcaataactactgcttagaaatatttagattaatatttcatttgcaacaataagaatggcagaaggatttgcagccaatagacaatggataggactagaagaagctgaagagttattagattttgatatagcaacacaaatgagtgaagaaggaccactaaatccaggagtaaacccatttagggtacctggaataacagaaaaagaaaagcaaaactactgtaacatattacaacctaagttacaagatctaaggaacgaaattcaagaggtaaaactggaagaaggaaatgcaggtaagtttagaagagcaagatttttaaggtattctgatgaaagtgtattgtccctggttcatgcgttcataggatattgtatatatttaggtaatcgaaataagttaggatctttaagacatgacattgatatagaagcaccccaagaagagtgttataataatagagagaagggtacaactgacaatataaaatatggtagacgatgttgcctaggaacggtgactttgtacctgattttatttataggaataataatatattcacagacaaccaacgctcaggtagtatggagacttccaccattagtagtcccagtagaagaatcagaaataattttttgggattgttgggcaccagaagaacccgcctgtcaggactttcttggggcaatgatacatctaaaagctaagacaaatataagtatacgagagggacctaccttggggaattgggctagagaaatatgggcaacattattcaaaaaggctactagacaatgtagaagaggcagaatatggaaaagatggaatgagactataacaggaccatcaggatgtgctaataacacatgttataatgtttcagtaatagtacctgattatcagtgttatttagatagagtagatacttggttacaagggaaaataaatatatcattatgtctaacaggaggaaaaatgttgtacaataaagttacaaaacaattaagctattgtacagacccattacaaatcccactgatcaattatacatttggacctaatcaaacatgtatgtggaatacttcacaaattcaggaccctgaaataccaaaatgtggatggtggaatcaaatggcctattataacagttgtaaatgggaagaggcaaaagtaaagtttcattgtcaaagaacacagagtcagcctggatcatggtttagagcaatctcgtcatggaaacaaagaaatagatgggagtggagaccagattttgaaagtaaaaaggtgaaaatatctctacagtgcaatagcacaaaaaacctaacctttgcaatgagaagttcaggagattatggagaagtaacgggagcttggatagagtttggatgtcatagaaataaatcaaaacttcatgctgaagcaaggtttagaattagatgtagatggaatgtagggagtaatacctcgctcattgatacatgtggaaacactcaaaaagtttcaggtgcgaatcctgtagattgtaccatgtattcaaataaaatgtacaattgttctttacaaaacgggtttactatgaaggtagatgaccttattatgcatttcaatatgaaaaaggctgtagaaatgtataatattgctggaaattggtcttgtacatctgacttgccatcgtcatgggggtatatgaattgtaattgtacaaatagtagtagtagttatagtggtactaaaatggcatgtcctagcaatcgaggcatcttaaggaattggtataacccagtggcaggattacgacaatccttagaacagtatcaagttgtaaaacaaccagattacttagtggtcccagaggaagtcatggaatataaacctagaaggaaaagggcagctattcatgttatgttggctcttgcagcagtattatctattgccggtgcagggacgggggctactgctatagggatggtaacacaataccaccaagttctggcaacccatcaagaagctgtagaaaaggtgactgaagccttaaagataaacaacttaagattagttacattagagcatcaagtactagtaataggattaaaagtagaagctatggaaaaatttttgtatacagctttcgctatgcaagaattaggatgtaatcaaaatcaatttttctgcaaaatccctcctgagttgtggacaaggtataatatgactataaatcaaacaatatggaatcatggaaatataactttgggggaatggtataaccaaacaaaagatttacaacaaaagttttatgaaataataatggacatagaacaaaataatgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaatcaaactaataaagtatgtattgtaaggtaaaaggaaaagacaaagaagaagaagaaagaagaaagccttcaagaggatgatgacagagttagaagatcgcttcaggaagctatttggcacgacttctacaacgggagacagcacagtagattctgaagatgaacctcctaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgca

SEQ ID NO: 2 - DNA, 20 bp, Artificial
  Oligonucleotide
ttaaccgcaaaaccacatcc

SEQ ID NO: 3 - DNA, 25 bp, Artificial
  Oligonucleotide
tgaaccctgtcgtgtatctgtgtaa

SEQ ID NO: 4 - DNA, 21 bp, Artificial
  Oligonucleotide
agataccatgctctacactgc

SEQ ID NO: 5 - DNA, 19 bp, Artificial
  Oligonucleotide
aacatggggaatggacagg

SEQ ID NO: 6 - DNA, 21 bp, Artificial
  Oligonucleotide
ggtatatcaccaggttctcgt